(19) United States
(12) Patent Application Publication
(10) Pub. No.: US 2004/0058428 A1
(43) Pub. Date: Mar. 25, 2004

(54) Selective growth medium for Bacillus anthracis and methods of use

(76) Inventors: Daniel Perlman, Arlington, MA (US); Inga Mahler, Newton, MA (US)

Correspondence Address: PERKINS, SMITH & COHEN LLP, ONE BEACON STREET, 30TH FLOOR, BOSTON, MA, 02108, US

(21) Appl. No.: 10/366,244

(22) Filed: Feb. 13, 2003

Related U.S. Application Data
(60) Provisional application No. 60/356,510, filed on Feb. 13, 2002.

(51) Int. Cl. C12N 1/20

(52) U.S. Cl. 435/252.3

(57) ABSTRACT

A solid nutrient medium that is selective for culturing and detecting B. anthracis bacteria and spores is described herein. This culturing and detection is based upon formation of B. anthracis colonies on the nutrient medium after incubation at room temperature or at elevated temperature. The medium of various embodiments includes a rich nutrient medium capable of supporting the growth of B. anthracis and the following constituents: a nutrient medium gelling agent, an anti-fungal agent for suppressing the growth of mold, a thallous salt, an EDTA salt, lysozyme, and a gram negative antibacterial agent including a cephalosporin compound, all at concentrations insufficient to inhibit the growth of B. anthracis.

### RELATED APPLICATIONS

This patent application claims priority to U.S. Provisional Application Serial No. 60/356,510, filed Feb. 13, 2002.

### TECHNICAL FIELD

The present invention relates to a bacterial growth medium designed for selectively growing Bacillus anthracis.

### BACKGROUND OF THE INVENTION

Detection of Bacillus anthracis (hereinafter abbreviated B. anthracis) has become an important issue in light of recent events concerning bioterrorism. An effective diagnostic test should be specific and selective. However, existing tests appear to have a high incidence of false positives and false negatives which necessitates performing duplicate tests, a requirement that can be time consuming and inefficient. For example, hand held or smart-ticket assays can be used in the field and are primarily designed for military use. These tests produce a high incidence of false positives and false negatives and are only sensitive enough to detect a minimum of 8000-10,000 anthrax spores. An alternative is a PCR based diagnostic test. The test which detects unique regions of the anthrax genome generally requires a clinical laboratory and trained technicians. Other clinical assays include immunological assays such as enzyme-linked immunosorbent assay (“ELISA”) or electrophoretic immunotransblot assays for analyzing clinical specimens.

Previous microbiological detection methods have also been described, employing selective nutrient media that allow B. anthracis to form colonies while restricting the growth of some other species. For example, Pearce et al. (J. gen. Microbiol. 5, 387-390, 1951) describe a nutrient medium containing lysozyme and hematin. Many other species of Bacillus can form colonies on this medium when incubated at room temperature thereby limiting the utility of this medium. Morris (J. gen. Microbiol. 13, 456-460, 1955) describes a peptone agar medium containing packed red cells, propamidine isothionate and polymyxin B. Besides allowing Proteus vulgaris and B. cereus to form colonies in addition to B. anthracis, the utility of this medium is limited owing to its susceptibility to small changes in pH. With only a 0.4 pH unit increase from the specified pH of 7.6 to a pH of 8.0, the growth of B. anthracis was detectably inhibited. Knisely (J. Bacteriol. 92 784-786, 1966) describes a B. anthracis selective nutrient medium that has greater utility for detecting B. anthracis than the earlier developed media described above. However, the Knisely medium still has limitations that are explained in detail below.

Characteristics of B. anthracis, as well as tests for its presence and identity, are summarized in the Manual of Standards for Diagnostic Tests and Vaccines, 4^{th} Edition, 2000, Chapter 2.2.1, Anthrax, the entire contents of which are hereby incorporated by reference (see http://www.oie.int/eng/normes/MMANUAL/A_{—}00038.htm).

As used herein, the following terms shall have the meanings indicated, unless the context otherwise requires: An “elevated temperature” is a temperature above room temperature and below 100 degrees Centigrade, for example, 37 degrees Centigrade. A “Bacillus anthracis nutrient source” is a nutrient suitable for supporting the growth of Bacillus anthracis spores and vegetative cells.

Clearly, there is a current need for a selective growth medium that will support the growth of B. anthracis while limiting the growth of other microbiological species. Optimally, this medium would be used in a test system employed to detect B. anthracis.

### SUMMARY OF THE INVENTION

A solid nutrient medium that is selective for culturing and detecting B. anthracis cells and spores is described herein. This culturing and detection process is based upon formation of B. anthracis colonies on the nutrient medium after incubation at room temperature or at elevated temperature. The medium of various embodiments disclosed herein include, but is not limited to, a rich nutrient medium capable of supporting the growth of B. anthracis and the following constituents: a nutrient medium gelling agent, an anti-fungal agent for suppressing the growth of mold, a thallous salt, an EDTA salt, lysozyme, and a gram negative antibacterial agent including a cephalosporin compound, all at concentrations insufficient to inhibit the growth of B. anthracis.

In one embodiment of the invention there is provided a selective growth medium for B. anthracis comprising a nutrient source, and at least one gram-positive inhibitor at a concentration insufficient to prevent the growth of B. anthracis, at least one gram-negative inhibitor at a concentration insufficient to prevent the growth of B. anthracis, including a cephalosporin compound that can also be a partial or a significant inhibitor of gram positive cells.

In a further related embodiment, the cephalosporin compound is a second or third generation cephalosporin compound, and in specific embodiments is cefuroxime, cefotaxime, ceftazidime, ceftizoxime. Alternatively, or in addition, there are provided a plurality of gram negative and gram positive inhibitors in concentrations insufficient to prevent the growth of B. anthracis, and specific embodiments here include the gram positive inhibitor lysozyme at a final concentration of about 40 μg/mL, thallous acetate at a final concentration of between about 4 μg/mL and 40 μg/mL, and a chelating agent such as ethylenediaminetetraacetic acid (EDTA) at a final concentration of about 300 μg/mL. Alternatively, or in addition, one or more anti-fungal agents are added in concentrations insufficient to prevent the growth of B. anthracis but sufficient for suppressing the formation of mold colonies, some of which could be confused with B. anthracis in the absence of microscopic analysis. For example, cycloheximide can be added to the selective growth medium at a final concentration of about 100 μg/mL to prevent mold growth. Alternatively, or in addition, the nutrient source for the B. anthracis includes compositions selected from the group comprising a rich nutrient medium such as beef heart infusion. Alternatively, or in addition, the selective liquid growth medium can be solidified by agar or other gelling compounds. In addition, the medium has a pH of about 7.35.

In another embodiment of the invention, a kit is provided that includes a culturing vessel such as a Petri dish for containing a B. anthracis selective growth medium, the selective growth medium further comprising a nutrient source, a gram positive inhibitor at a concentration insufficient to prevent the growth of B. anthracis, and a gram negative inhibitor including a cephalosporin compound.

The kit can further comprise: (i) a sealable storage vessel or bottle for the less perishable liquid or gelled components of the selective growth medium, and (ii) a small vial, capsule or the like, that contains as dry and chemically stable ingredients, the balance of selective growth medium components. The latter components include those that would be perishable and degraded if dissolved, mixed and stored (e.g., at room temperature for a week or more) with the less perishable liquid or gelled components of the growth medium.

The kit can further comprise a temperature indicating strip, e.g., a liquid crystal flat strip, or other temperature indicating device, attached or attachable to the sealable storage vessel. This indicating device can be useful for example, during cooling of a re-melted agar gel-containing growth medium to determine when the temperature has sufficiently decreased to add the relatively perishable (and temperature-labile) dry ingredients.

In addition, the kit can include an applicator device or swab to aid in applying an environmental sample to the growth medium in the culturing vessel. The kit can further comprise a sealable outer container for inserting the vessel subsequent to application of the sample, suitable for incubating the sample and determining the presence of B. anthracis.

### DETAILED DESCRIPTION

Recent incidents of B. anthracis infections in the human population have increased the demand for reliable, yet versatile and cost-effective protocols (collectively termed “testing systems”) for detecting B. anthracis in field samples of bacteria obtained from a variety of locations and sources. Ideally, such testing systems should be suited for use in the field by unskilled personnel. The inventors decided to focus on developing a solid growth medium for cultivating B. anthracis that might be usable in the field with a minimum of equipment. They further developed the growth medium for use at room temperature thereby obviating the need for equipment such as incubators for sustaining an elevated temperature, e.g., 37° C.

Selective solid growth media for culturing bacteria have long provided an important low-cost, and reliable method for detecting pathogenic bacteria derived from the natural environment as well as from clinical settings. Once a single bacterium or spore finds its way onto a selective nutrient medium capable of sustaining its growth, the bacterium is generally capable of multiplying to form a colony, the colony being easily detected with the unaided eye. The identity of the bacterium can be further identified by its appearance and other physical and biochemical properties. A problem presented by this approach is creating a medium which is indeed selective for a particular bacterial species. This medium should discourage the growth of microbiological species, e.g., bacteria and mold species, other than the target species. The medium should further provide sufficient nutrients to permit colony formation within a reasonable period of time, for example, 1-3 days.

B. anthracis is a type of spore-forming gram positive bacterium that traditionally has been difficult to differentially culture and distinguish from other microorganisms, e.g., other Bacilli outside of controlled laboratory conditions.

In the mid 1960s a selective growth medium was developed that provided limited success in selectively growing B. anthracis in culture (Knisely, J. Bacteriol. 92, 784-786, 1966, the entire contents of which are herein incorporated by reference). B. anthracis was grown by Knisely at 37° C. in a rich growth medium known as PLET, containing beef heart infusion agar; polymyxin (30 U/mL final); lysozyme (40 μg/mL final); disodium ethylenediaminetetraacetate (EDTA—300 μg/mL final); and thallous acetate (40 μg/mL final) at a final pH of 7.35. Unfortunately, in recent decades, a variety of bacteria have become resistant to inhibitors in the PLET medium resulting in loss of specificity for B. anthracis. In fact, the PLET medium allows growth of a number of common gram positive and gram negative bacterial species including B. subtilis, Streptococcus faecalis, Staphylococcus albus, Staphylococcus aureus and Escherichia coli. Furthermore, with design of the PLET medium it was not appreciated that use of this medium in the field, e.g., testing indoor air for the presence of B. anthracis, is compromised by the presence of airborne mold spores, which are abundant almost everywhere in the environment. These mold spores can readily germinate and grow on the PLET medium when incubated either at room temperature or at 37° C. to produce visible colonies within the same time period (1-3 days) allotted for B. anthracis colony appearance. The growth of other bacteria as well as molds, i.e., fungal species, on the PLET medium that is intended to detect B. anthracis diminishes the value of the medium as a diagnostic and screening tool.

A diagnostic nutrient medium for B. anthracis that is sensitive, reliable, low cost and easy to use by unskilled personnel both in a clinical laboratory setting and in the field is disclosed herein. In particular, a selective solid growth medium that favors the growth of B. anthracis at room temperatures and higher temperatures, e.g., 37° C., is disclosed. The medium contains inhibitors of growth of gram negative and gram positive bacteria, and, in various embodiments, at least one inhibitor of molds. Thus, a selective medium and method of use is provided that allows for the reliable screening of indoor air, hard and soft surfaces such as postal mail, suspect powders, and other materials originating from a variety of locations and sources for the presence of B. anthracis. The medium could optionally include other identifying agents to facilitate detection of B. Anthracis.

The solid growth medium is an aqueous medium that is gelled with agar. Alternatively, the solid growth medium can be prepared using other gelling hydrocolloid polymer materials including purified agaroses (Hispanagar S. A., Burgos, Spain) such as standard melting temperature agaroses (refined from agar), low melting temperature agaroses (chemically modified by, for example, hydroxyethylation), xanthan plus galactomannan binary gels, and other microbiological media gelling systems. The resulting solidified growth media incorporates a rich nutrient source such as beef heart infusion. The medium further contains at least one gram negative bacteriological inhibitor, and in particular, a second or third generation cephalosporin such as cefotaxime and at least one gram positive inhibitor such as lysozyme. In addition to lysozyme, thallous acetate and a chelating agent such as EDTA appear to be valuable for inhibiting gram positive bacteria including various Bacilli other than B. anthracis. In addition, the presence of an anti-fungal agent such as cycloheximide can be important for suppressing the formation of mold colonies, some of which could be confused with B. anthracis in the absence of microscopic analysis. The medium is preferably formulated in the neutral pH range between 6 and 8.5, and preferably around pH 7. One preferred medium, described below, is formulated without pH adjustment has a pH of approximately 7.35.

The components of one preferred B. anthracis-selective solid medium are all commercially available, and can be conveniently stored as two ready-to combine mixtures. One is a gelled aqueous growth medium, and the other is a dry antibiotic powder blend. The gelled medium and the dry antibiotic blend both have excellent shelf-stability when stored separately (shelf life of 1-2 years). When, for example, one Petri dish of B. anthracis-selective medium is needed for B. anthracis screening, the gelled sterile growth medium stored in a 0.75-1 ounce HDPE plastic screw capped bottle (e.g., 15-20 mL of Heart Infusion Agar (Difco Company) supplemented with disodium EDTA (300 μg per mL) and thallous acetate (8 μg per mL)) is melted, i.e., liquefied, by dropping the bottle into a saucepan of boiling water for 5 minutes. The bottle is then cooled in air at room temperature for 9-12 minutes after which the temperature of the medium has dropped to 50-55° C. A thin and inexpensive, yet accurate liquid crystal-type thermometer strip graduated in 5° C. increments between 30 and 60° C., adhered to the outside surface of the bottle allows convenient temperature monitoring (Hallcrest Inc., Glenview Ill.). At approximately 50° C., the temperature-labile antibiotic-containing powder blend (stored in a capsule, pouch, or small vial) can be safely added and dissolved in the warm medium by gently inverting the bottle a few times. In the presently described preferred medium, the antibiotic powder provides final concentrations in the medium of lysozyme (40 μg/mL), cefotaxime (5 μg/mL) and cycloheximide (100 μg/mL). The now complete B. anthracis selective medium is finally poured into a sterile plastic disposable Petri dish where it cools, solidifies, and is ready for use. The poured Petri plates can be wrapped up to prevent drying, and stored in a refrigerator for up to several weeks time if they are not needed for immediate use. Experience has show that after inoculating the selective plates with a B. anthracis-containing bacterial inoculum, visible B. anthracis colonies typically appear overnight with 37° C. incubation, or after approximately 28-48 hours with incubation at 22° C. Sterile disposable Dacron polyester swabs have been used to sample hard and soft surfaces including counter tops, laboratory benches, postal envelopes, other field samples, and even human epithelial surfaces, e.g., nasal passages. These sterile swabs are usefully packaged and included in a test kit that can also include the above-described Petri dish and B. anthracis selective medium. As described above, this culturing medium can be provided in the form of ready-to-mix components or alternatively, in the form of pre-poured plates that are ready to inoculate with suspected B. anthracis contaminant samples. The pre-poured plates are preferably stored in such a manner to prevent or substantially reduce water loss through evaporation. Petri dishes can be wrapped with sealing tape around their circumference or bagged to achieve similar results.

In addition to laboratory use of the B. anthracis selective medium in which the medium is supplied in standard Petri dishes to the user in standard packaging, the preparation of easy-to-use individualized field kits that fit into a coat pocket is disclosed. Each kit contains ready-to-use pre-poured solidified medium packed in such a way as to avoid desiccation of the medium. For example, a screw-capped tube can be provided with a label for describing the sample, in which the tube contains solidified medium formed as a slant (solidified on a slope of about 30°-45° to the walls of the cylindrical tube). Accordingly, each kit would optionally include a small sterile screw-capped bottle of plastic or glass containing the growth medium, a sterile specimen applicator device such a spreader or swab for applying the suspect sample to the Petri dish or to the slant of B. anthracis selective nutrient medium. The kit can further include a receptacle for containing and sealing the Petri dish or tube after the sample has been applied and additionally a receptacle for receiving the used spreader. The kit can additionally contain disposable plastic gloves and instructions for use of the kit.

### EXAMPLES

### Example 1

### Prior Art Selective Growth Media

The prior art PLET selective solid growth medium for B. anthracis was prepared according to Knisely (1966) comprising Heart Infusion Agar (Difco), the gram positive inhibitor lysozyme at a final concentration of 40 μg/mL, the gram negative inhibitor polymyxin (polymyxin B sulfate) at 30 μg/mL, disodium EDTA at 300 μg/mL and thallous acetate at 40 μg/mL. The pH of the selective growth medium was about 7.35. This medium which was placed in Petri dishes and incubated at 37° C. was tested against 13 diverse bacterial stock species. B. anthracis colonies appeared after overnight incubation. The growth of other bacilli including B. megaterium, B. polymyxa, B. pumilus and B. subtilis was prevented. The growth of several B. cereus strains was reduced. On identical plates of the same medium exposed to indoor air for 15 minutes and then incubated at either 37° C. or 20° C., a variety of random mold colonies appeared within 24-48 hours.

### Example 2

### Addition of Cycloheximide

A growth medium is described that is selective for B. anthracis, and that is similar to Example 1 above except that it additionally contains the mold inhibitor, cycloheximide at a final concentration of about 100 μg/mL. This concentration of cycloheximide did not alter the pattern of bacterial growth at either 37° C. or 20° C., but eliminated all mold contamination. This feature is important to the utility of the selective medium because in the absence of cycloheximide, airborne mold spores germinated and formed colonies within approximately the same time frame required for B. anthracis colony formation. In the absence of microscopic examination, some of these mold colonies (often white to gray-white smooth colonies) could be confused with B. anthracis colonies. Such mold colonies would be “false positives” and if abundant, could cause unnecessary alarm. Various mold inhibitors including clotrimazole, nystatin, and amphotericin B can be considered as alternatives to cycloheximide in this applied use. Optimal concentrations for each antimycotic agent in the B. anthracis selective media can be determined by routine experimentation.

### Example 3

### Thallous Acetate Effect

Knisely (1966) suggests that despite the known toxicity of thallium ion and its ability to inhibit the growth of a variety of microorganisms, the B. anthracis species can be exceptional is this regard. While B. anthracis is more resistant to thallium than many other bacterial species, the 40 μg/mL level that Knisely specifies is quite inhibitory to B. anthracis growth as well (appearance of visible colonies is delayed approximately one day compared to using the 4 μg/mL level). Accordingly, a selective growth medium for B. anthracis similar to Example 2 above was formulated, except that the thallous acetate concentration was varied between 4 μg/mL and 40 μg/mL. The 4 μg/mL level was not sufficiently restrictive to prevent growth of non-anthracis gram positive species including some strains of B. cereus that have resistance to low levels of thallous acetate. While 40 μg/mL thallous acetate retarded B. anthracis growth excessively, growth with 6-8 μg/mL thallous acetate was considerably faster. In fact, 8 μg/mL thallous acetate appeared to be ideal since it proved to be adequately restrictive to non-anthracis gram positive spore forming species (including some B. cereus strains) while it allowed B. anthracis to grow readily and form colonies. Interestingly, some gram positive bacterial species including Staphylococcus aureus appeared to be completely resistant to thallous acetate, growing just as rapidly with 40 μg/mL thallous acetate as with no thallous acetate.

### Example 4

### Effect of Increasing the EDTA Level and Addition of Lysostaphin

A series of selective growth media for B. anthracis were formulated similar to Example 3 above, containing 8 μg/mL thallous acetate and 100 μg/mL cycloheximide (in addition to 40 μg/mL lysozyme and 300 μg/mL disodium EDTA and 30 μg/mL polymyxin). Doubling the EDTA concentration or both the EDTA and the lysozyme concentrations had no effect on Staphylococcus aureus growth, while undesirably inhibiting the growth of B. anthracis. However, maintaining the original levels of EDTA and lysozyme and adding between 0.5 units and 12.5 units/mL of the enzyme, lysostaphin (Sigma Chemical) severely retarded the growth of Staphylococcus aureus without affecting B. anthracis growth. Therefore, lysostaphin can be utilized in this type of B. anthracis selective medium to inhibit Staphylococcus aureus growth.

### Example 5

### Inhibiting the Growth of Gram Negative Bacteria

A series of selective growth media for B. anthracis were formulated similar to Example 3 above (containing 8 μg/mL thallous acetate, 100 μg/mL cycloheximide, 40 μg/mL lysozyme and 300 μg/mL disodium EDTA). Polymyxin was eliminated from the formulation since it had proven ineffective as a gram negative inhibitor, e.g., growth of three out of four strains of E. coli was not inhibited. It is likely that the over-use of polymyxin during the past 30 years has resulted in many gram negative species developing a resistance to this antibiotic.

First, streptomycin was added to the above formulation at a concentration of 25 μg/mL. Streptomycin is bactericidal against aerobic gram negative bacilli and some mycobacteria. In fact, all of the available E. coli strains were inhibited by streptomycin.

Next to be tried was the combination of trimethoprim and sulfamethoxazole (known commercially as Bactrim® that inhibits the synthesis of tetrahydrofolic acid). This experiment followed many reports in the literature that Bactrim® was ineffective in treating anthrax infections but very effective for inhibiting the growth of aerobic gram negative bacteria. The susceptible species include E. coli, Proteus mirabilis, Salmonella, Shigella, Citrobacter, Haemophilis influenzae, Vibrio cholerae, Yersinia pestis, Acinetobacter, Bordetella pertussis, Brucella and some Pseudomonas species, as well as inhibiting some gram positive species as well, including Staphylococcus aureus, Streptococcus pneumoniae and S. pyrogenes. Accordingly, trimethoprim and sulfamethoxazole were added to the above formulation (see Example 5) at concentrations of 2 μg/mL and 40 μg/mL respectively. While this B. anthracis selective medium allowed B. anthracis colonies to grow normally, there was only a diminution rather than a fully effective inhibition of three different gram negative strains including Klebsiella pneumoniae, Citrobacter freundii, and E. coli. Also, the limited solubility of these chemicals in the bacteriological culture medium resulted in a suspension of these chemicals rather than a solution. Because of the broad spectrum of bacterial species inhibited while B. anthracis growth remains uninhibited, the combination of these two antibiotics is considered potentially useful in spite of the solubility matter.

### Example 6

### Inhibiting the Growth of Gram Negative and Positive Bacteria with Cephalosporins

A series of selective growth media for B. anthracis were formulated similar to Example 5 above (containing 8 μg/mL thallous acetate, 100 μg/mL cycloheximide, 40 μg/mL lysozyme and 300 μg/mL disodium EDTA but again without polymyxin). In this Example, a cephalosporin was used, and more specifically, a third generation cephalosporin known as cefotaxime was utilized. The cephalosporins are beta-lactam antibiotics that primarily act like penicillin as bactericidal agents by binding to one or more penicillin binding proteins (PBPs), e.g., beneath the cell wall. However, the first generation cephalosporins (C-1) have greater affinity for PBPs of gram positive Staphylococci while second and third generation cephalosporins (C-2 and C-3) have greater affinity for PBPs of the gram negatives, e.g., enteric bacteria such as E. coli. Thus, C-1 antibiotics are most useful for killing gram positive bacteria, while C-2 and C-3 antibiotics are mainly used for killing gram negatives. In fact, at least three of the C-1 antibiotics including cefazolin, cephalothin, and cephradine have been listed in the literature as being potential treatments for B. anthracis infections. Therefore these would be unsuitable for use in the present invention. On the other hand, cefuroxime (C-2), cefotaxime (C-3), ceftazidime (C-3) and ceftizoxime (C-3) are cephalosporins that have been reported as ineffective for treatment of B. anthracis infections.

Given the above, use of C-2 and C-3 antibiotics can allow B. anthracis growth and diagnostic colony appearance on the selective media of the present invention, while inhibiting or killing non-anthracis bacteria. One of the more readily available C-3 antibiotics that is available as a generic drug is cefotaxime. Cefotaxime sodium is readily dissolved in the aqueous nutrient media described in the present invention. This antibiotic is described (Drug Evaluations, 6th Edition; American Medical Association, Chicago, Ill.) as effective against a wider spectrum of gram negative bacteria than C-1 and C-2 antibiotics due to its resistance to beta-lactamases and its higher affinity for many of the gram negative bacterial penicillin binding proteins. In addition, a significant number of gram positive bacterial species including a variety of streptococcal species and most staphylococcal species are susceptible to cefotaxime. If this would prove true at antibiotic levels that still would allow B. anthracis colonies to appear, this antibiotic would be very valuable in improving the B. anthracis selective culture medium.

When cefotaxime is administered to humans, peak serum concentrations reach levels of between 20 and 100 μg/mL. Considering that B. anthracis has been described as resistant to cefotaxime, 20, 40 and 60 μg/mL of cefotaxime was added to the B. anthracis selective medium described at the beginning of this Example 6. Surprisingly, B. anthracis bacterial inocula on this medium were sensitive rather than resistant to the cefotaxime. In fact, colonies failed to appear on the 40 and 60 μg/mL containing plates even after 3 days, and appeared with 20 μg/mL cefotaxime only after 2-3 days incubation at 37° C. (rather than within 24 hours for control plates containing the B. anthracis selective medium without cefotaxime. After successive trials it was discovered that only very low levels of cefotaxime (e.g., in the range of between approximately 2 and 8 μg/mL, and most preferably 3-5 μg/mL) would allow reasonably rapid appearance of B. anthracis colonies (1 day when incubated at 37° C., and 2 days when incubated at 20° C.) on the B. anthracis selective media described in this Example. The unanticipated inhibition of B. anthracis by higher levels of cefotaxime (a third generation cefalosporin) might be caused by the simultaneous presence of (and synergistic inhibition by) the other gram positive inhibitors including lysozyme.

Remarkably, even with these very low levels of cefotaxime included in the selective medium, a wide variety of gram negative bacteria, e.g., E. coli, Klebsiella pneumoniae, Citrobacter freundii, and gram positive species, e.g., both streptococcal and staphylococcal species, e.g., Staphylococcus aureus, that were tested were inhibited. Many of the non-anthracis species of the genus Bacillus that were tested, including B. subtilis, B. megaterium, and B. thuringiensis were also inhibited. Some strains of B. cereus that were available for testing did, in fact, grow to form colonies within the same incubation time period as B. anthracis strains. Therefore, the present medium does not immediately distinguish between these two species based upon growth alone. However, subsequent testing of the emerging colonies can distinguish between these species. Third party testing has confirmed that a medium in accordance with this Example exhibits valuable selectivity for a variety of B. anthracis strains.

### Example 7

### Composition for a B. anthracis Selective Medium and Design of Kit

A useful B. anthracis selective medium consists of Heart Infusion Agar (Difco) to which is added 8 μg/mL thallous acetate, 300 μg/mL disodium EDTA, 100 μg/mL cycloheximide, 40 μg/mL lysozyme and between approximately 2 and 8 μg/mL cefotaxime (e.g., 5 μg/mL cefotaxime). The selective growth medium is agar-based, and has a pH of between 7 and 8 (e.g., about 7.35). The selective growth medium includes a nutrient source (i.e., Heart Infusion Agar), gram positive bacteriological inhibitors, (i.e., lysozyme, EDTA, thallium acetate, and cefotaxime in an amount insufficient to prevent the growth of B. anthracis), and at least one gram negative bacteriological inhibitor (i.e., the same cefotaxime antibiotic used as a gram positive inhibitor) and a mold inhibitor (i.e., cycloheximide). For preparing pre-poured plates containing the B. anthracis selective medium, the above medium can be autoclaved without the heat-labile components (i.e., without cycloheximide, lysozyme and cefotaxime). Following autoclaving, as the medium cools to approximately 50-55° C., the heat-labile components can be added as a filter-sterilized aqueous solution, or alternatively as a dry powder which is dissolved with gentle mixing. Approximately 15-20 mL of the combined medium is poured into each 100 mm diameter Petri dish.

Alternatively, an easy-to-use kit has been designed that utilizes the same B. anthracis selective medium composition but provides greater shelf stability than pre-poured plates. This kit allows the user to pour their own Petri plates when they are needed. The kit includes: (i) one or more empty sterile 100 mm diameter Petri dishes for holding approximately 15-20 mL of the growth medium; (ii) a sealed high density polyethylene or polypropylene storage bottle (¾-1 ounce capacity) containing, as a sterile gel, the less perishable gelled liquid components of the selective growth medium, i.e., the Heart Infusion Agar, EDTA and thallium acetate and (iii) a small capsule or vial that contains, as a dry mixture, the balance of selective growth medium components, i.e., the lysozyme, cefotaxime, and cycloheximide The latter components are those that would become degraded if dissolved, mixed and stored for a period of time before use (e.g., at room temperature for a week or more). A temperature-indicator e.g., a flat liquid crystal thermometer strip (manufactured by Hallcrest, Inc, Glenview, Ill.) is attached to the storage bottle. This thermometer device is used during cooling of the re-melted agar-containing growth medium to show when the medium has cooled sufficiently, i.e., to 50-55° C., to allow addition and dissolving of the temperature-labile dry ingredients. When all ingredients have been added and dissolved, the liquid is poured into the Petri dish and cooled. The poured Petri dish can be used immediately or can be wrapped to prevent water evaporation and stored refrigerated for a number of weeks. In addition, the kit can include an applicator device or swab to aid in applying an environmental sample to the growth medium in the culturing vessel. The kit can further comprise a sealable outer container for inserting the vessel subsequent to application of the sample, suitable for incubating the sample and determining the presence of B. anthracis.

### Example 8

### Method of Utilizing B. anthracis Selective Medium

A bacterial sample is collected from a location or source such as a swab wipe from a postal mailing envelope, a public desk surface or a swab from a human epithelial surface, or a bodily fluid. The bacterial sample is transferred from the swab, or via any other application method to a growth medium selective for B. anthracis, such as that of Example 6, by spreading the sample over an agar surface of a Petri plate, for example. Any B. anthracis present in the sample is allowed to grow at 37° C. or at room temperature for 24-48 hours. The cultures are monitored for the presence of B. anthracis colonies, which appear white or grey-white and are about 0.3-2 mm in diameter. Positive identification of B. anthracis is then carried out in specified diagnostic laboratories utilizing, for example, PCR DNA amplification and/or immunological methods.

What is claimed is:

1. A solid nutrient medium that is selective for culturing and detecting B. anthracis bacteria and spores based upon formation of visible B. anthracis colonies on said solid nutrient medium after incubation at room temperature or at elevated temperature, comprising a heart infusion nutrient medium and effective concentrations of the following constituents: a gelling agent, an anti-fungal agent for suppressing the formation of mold colonies, a thallous salt, a chelating agent, a gram positive antibacterial agent at a concentration insufficient to prevent growth of B. anthracis bacteria, and a gram negative antibacterial agent including a cephalosporin compound.

2. The solid nutrient medium of claim 1 wherein the final pH of said solid nutrient medium is between pH 6.0 and pH 8.5.

3. The solid nutrient medium of claim 2 wherein the final pH of said solid nutrient medium is between pH 7.0 and pH 7.7.

4. The solid nutrient medium of claim 1 wherein said gelling agent is selected from the group consisting of agar and agarose.

5. The solid nutrient medium of claim 1 wherein said thallous salt is selected from the group consisting of thallous acetate, thallous carbonate and thallous chloride.

6. The solid nutrient medium of claim 1, wherein the chelating agent is an EDTA salt.

7. The solid nutrient medium of claim 1 wherein said EDTA salt is selected from the group consisting of disodium EDTA, dipotassium EDTA and mixed salts and combinations thereof.

8. The solid nutrient medium of claim 6, wherein said cephalosporin compound is selected from the group consisting of cefuroxime, cefotaxime, ceftazidime and ceftizoxime.

9. The solid nutrient medium of claim 1 wherein said cephalosporin compound is selected from the group consisting of second generation cephalosporin compounds, third generation cephalosporin compounds and combinations thereof.

10. The solid nutrient medium of claim 9 wherein said cephalosporin compound is selected from the group consisting of cefuroxime, cefotaxime, ceftazidime and ceftizoxime.

11. The solid nutrient medium of claim 10 wherein said cephalosporin compound is cefotaxime.

12. The solid nutrient medium of claim 1 wherein said anti-fungal agent is selected from the group consisting of clotrimazole, nystatin, amphotericin B and cycloheximide.

13. A medium that is selective for culturing and detecting B. anthracis bacteria and spores, at least one of room temperature or an elevated temperature, comprising a Bacillus anthracis nutrient source, a gram positive antibacterial agent at a concentration insufficient to prevent growth of Bacillus anthracis, and a gram negative antibacterial agent including a cephalosporin compound.

14. The medium of claim 13 wherein the final pH of the medium is between pH 6.0 and pH 8.5.

15. The medium of claim 14 wherein the final pH of said medium is between pH 7.0 and pH 7.7.

16. The medium of claim 13, further comprising a gelling agent, so that the medium is solid.

17. The medium of claim 16 wherein the gelling agent is selected from the group consisting of agar and agarose.

18. The medium of claim 13, further comprising a thallous salt.

19. The medium of claim 18 wherein the thallous salt is selected from the group consisting of thallous acetate, thallous carbonate and thallous chloride.

20. A kit for detecting the presence of B. anthracis bacteria or spores in an indoor or outdoor environment, wherein said kit comprises a first sealable container containing a gelled growth medium for supporting the growth of B. anthracis bacteria or spores, a second sealable container containing a dry blend of antibiotics comprising lysozyme, and at least one gram negative antibacterial agent including a cephalosporin compound for inhibiting the growth of non-anthracis microorganisms, and optionally at least one of an empty sterile Petri dish, a re-closable storage bag for said Petri dish, a sterile swab or applicator, and a thermometer.
